(19) United States
(12) Patent Application Publication
(10) Pub. No.: US 2011/0165674 A1
(43) Pub. Date: Jul. 7, 2011

(54) NK-1 Receptor Mediated Delivery of Agents to Cells

(75) Inventors: Anthony Kossiakoff, Chicago, IL (US); Shahir Rizk, Chicago, IL (US)

(73) Assignee: THE UNIVERSITY OF CHICAGO, Chicago, IL (US)

(21) Appl. No.: 12/797,137

(22) Filed: Jun. 9, 2010

Related U.S. Application Data
(63) Continuation of application No. PCT/US08/86274, filed on Dec. 10, 2008.
(60) Provisional application No. 61/012,514, filed on Dec. 10, 2007.
(60) Provisional application No. 61/114,221, filed on Nov. 13, 2008.

(51) Int. Cl. C12N 5/09; C07K 19/00; C07K 7/08

(52) U.S. Cl. 435/368; 530/391.1; 530/327; 530/322; 530/350

(57) ABSTRACT

Provided herein are conjugates including a targeting vehicle coupled to an agent. The targeting vehicle includes a tachykinin receptor ligand and a reactive moiety. Conjugates including a tachykinin receptor ligand attached to an antibody or fragment thereof that is specific for an intracellular target are also provided. Also provided are methods of delivering agents to cells expressing tachykinin receptors, methods of delivering antibodies or fragments thereof to an intracellular extra-endosomal target, and methods of arresting cell growth or introducing cell death of a cancer cell.

### CROSS-REFERENCE TO RELATED APPLICATIONS

This application claims priority to U.S. Provisional Patent Application Ser. No. 61/012,514, filed on Dec. 10, 2007 and U.S. Provisional Patent Application Ser. No. 61/114,221, filed on Nov. 13, 2008, both of which are incorporated herein by reference in their entireties.

### INTRODUCTION

The inability to introduce active proteins into cells has been a major bottleneck to understanding and affecting intracellular processes. Since many of the key components in a multitude of biological pathways are located within the cytoplasm, studying these pathways has to be carried out in either lysed or permeablized cells. In both cases, the cellular integrity is compromised and many of the key complexes that need to be identified d and characterized are destroyed or relocated. Additionally, the in vivo use of many therapeutic agents has been greatly limited due to the absence of methods for efficient and specific drug delivery to diseased tissue.

While some techniques are available for he intracellular, extra-endosomal or cytoplasmic delivery of proteins to living cells, most are met with major challenges. For example, the use of transfection reagents requires days for the target protein to be expressed and compromises the integrity of the membrane, in most cases leading to toxicity and limited in vivo utility. While cell-permeable peptides provide rapid delivery, they lack cell-type specificity and their cargo can be trapped in the endosome rendering it inactive. As a result of the inability of such delivery methods to selectively target diseased tissue, the therapeutic use of many reagents such as antibodies has been limited to cell-surface targets.

### SUMMARY OF THE INVENTION

Methods and compositions for delivery of agents to intracellular locations, or in some embodiments extra-endosomal locations, in cells expressing tachykinin receptors (NK-1, NK-2 or NK-3) are provided herein. The compositions and methods allow delivery of diverse agents across the cell membrane by a receptor-mediated delivery mechanism involving binding of an engineered peptide ligand, P (e.g., a Substance P variant) to a tachykinin receptor on the cell surface.

Conjugates for intracellular delivery of agents comprising a targeting vehicle conjugated to an agent are provided herein. The agent is suitably delivered to an intracellular extra-endosomal location. The conjugate has the structure: P-M-C-A. P is a tachykinin receptor ligand or a variant thereof. M is a reactive moiety attached to or near the N-terminus of P. C is an optional crosslinker. A is an agent attached to M or C, if present. Suitably M is maleimide and A is attached to M via a thioester bond.

In another aspect, conjugates for intracellular delivery of an antibody or fragment thereof comprising a targeting vehicle conjugated to an antibody or fragment thereof are provided herein. The antibody or fragment thereof is suitably specific for an intracellular target. The conjugate has the structure: P-M-C-Ab. P is a tachykinin receptor ligand or a variant thereof. M is a reactive moiety attached at or near the N-terminus of P. C is an optional crosslinker. Ab is an antibody or fragment thereof attached to M or C, if present.

In yet another aspect, methods of delivering an agent to an intracellular location within a cell expressing a tachykinin eceptor are provided. The methods include contacting the cell with a conjugate of the invention.

In still another aspect, methods of delivering an agent to a cell are provided. A polynucleotide encoding a tachykinin receptor operably connected to a promoter functional in the cell is introduced into the cell. Then the cell is contacted with the conjugate to deliver the agent to the cell.

In a further aspect, methods of delivering antibodies or fragments thereof to a cell expressing a tachykinin receptor are provided. The cells are contacted with a conjugate which includes a targeting vehicle and an antibody or fragment of an antibody. Suitably, the antibody or fragment thereof is specific for an intracellular target in the cell in which it is delivered.

In a still further aspect, methods of arresting cell growth or inducing death of a cancer cell are provided. The cancer cell is contacted with a conjugate including a targeting vehicle and an agent. The agent is capable of arresting cell growth or inducing death of the cancer cell.

In yet another aspect, methods of delivering an agent to an intracellular, extra-endosomal location within a cell expressing a tachykinin receptor are provided. The cells are contacted with a conjugate having the formula: P-M-C-A. P is a tachykinin receptor ligand or a variant thereof. M is a reactive moiety attached at or near the N-terminus of P. C is an optional crosslinker. A is an agent attached to M or C, if present.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1a is a schematic representation of receptor-mediated delivery (RMD) of an agent, referred to in the Figure as “cargo.” A fluorescently-labeled “cargo” is attached to a tachykinin receptor ligand. Upon binding to the receptor, the “cargo” is internalized where it can carry out the desired function.

FIG. 1b is a schematic representation of a conjugate of the invention showing attachment of synthetic antibody fragments (sABs) to a targeting vehicle based on a Substance P peptide (SP) capable of binding NK-1. A surface cysteine mutation at position 121 of the sAB reacts with the maleimide moiety of an engineered SP variant. The amino acid sequence of SP is shown in the box (SEQ ID NO. 2).

FIG. 2 is a set of micrographs showing internalization of functional sABs. In all panels, DAPI staining is shown in blue. FIG. 2a is a micrograph of HeLa cells treated with 100 nM SP-Alexa555 (red), and stained with FITC-phalloidin (green). FIG. 2b is an enlargement of the box shown in FIG. 2a, showing actin structure is unaffected. FIG. 2c is a micrograph of cells after treatment with 20 nM cy5-labeled sAB4-SP conjugate (red), followed by staining with TRITC-phalloidin (green). FIG. 2d is an enlargement of the box shown in FIG. 2c, indicating depolymerization of actin filaments. FIG. 2e and FIG. 2f are micrographs of HeLa cells treated with 20 nM cy3 labeled sAB17-SP (green in e), followed by staining with FITC-phalloidin (green in f), showing colocaliztion of the sAB with phalloidin. FIG. 2g is a micrograph showing HeLa cells treated with 20 nM cy5-labeled sAB27-SP conjugate (red) followed by staining with TRITC-phallodin (green). FIG. 2h is an enlargement of the box in FIG. 2g, showing the effect of sAB27 on actin filaments.

FIG. 3 demonstrates differential expression of the NK-1 receptor as between glioblastoma cells and normal human astrocytes (NHA). FIG. 3a is a photograph of a Western blot showing NK-1 receptor expression in NHA compared to two glioblastoma cell-lines U87 and U373. FIG. 3b is a micrograph showing internalization of 100 nM SP-Alexa555 (red) by U87 cells followed by staining with DAPI (blue). FIG. 3c is a micrograph showing no internalization was observed after treatment of NHA with 100 nM SP-Alexa555.

FIG. 4 is a set of micrographs showing internalization of sABs by U87 glioblastoma cells. FIG. 4a is a micrograph showing U87 cells treated with 20 nM cy5-labeled sAB4-SP conjugate (red), followed by staining with TRITC-phalloidin (green). FIG. 4b is an enlarged view of the box in FIG. 4a, indicating severing of actin filaments. FIG. 4c is a micrograph showing treatment of U87 cells with 20 nM cy5-labeled sAB27-SP conjugate (red) followed by staining with TRITC-phallodin (green). FIG. 4d is a micrograph of untreated cells. The structure of the actin filaments in FIG. 4c is disrupted as compared with untreated cells in FIG. 4d. FIG. 4e is a micrograph showing TRITC-phallodin (green) of untreated U87 cells. FIG. 4f is a micrograph showing treatment of U87 cells with 20 nM cy5-labeled full length actin antibody-SP conjugate (red).

FIG. 5 shows the results of cell viability assays of U87 glioblastoma cells after treatment with sAB27-SP. FIG. 5a is a graph showing the cell viability 48 hours after treatment with sAB27, SP or sAB27-SP conjugate, 10 nM; left panel, 20 nM; right panel. FIG. 5b is a graph showing the time course of inhibition of cell proliferation by the sAB27-SP conjugate, 10 nM; open circles, 20 nM; closed circles.

FIG. 6 is a graph plotting quantification of internalized conjugate as a function of the amount of conjugate added to the U87 cells after incubation with varying concentrations of fluorescently labeled conjugate for 24 hrs.

FIG. 7 is a schematic representation displayed with exemplary micrographs showing the fluorescence of Alexa-647-labeled polynucleotide encoding GFP operably linked to a promoter (red) after internalization by cells after 2 hours. After 24 hours incubation, GFP expression is observed (green) within cells treated with the conjugate, but not in cells treated with the polynucleotide alone.

FIG. 8 is a schematic representation displayed with exemplary micrographs showing fluorescence of actin filaments (green) and Myosin (red) in cells after incubation with SP-GFP-Myosin VI.

### DETAILED DESCRIPTION

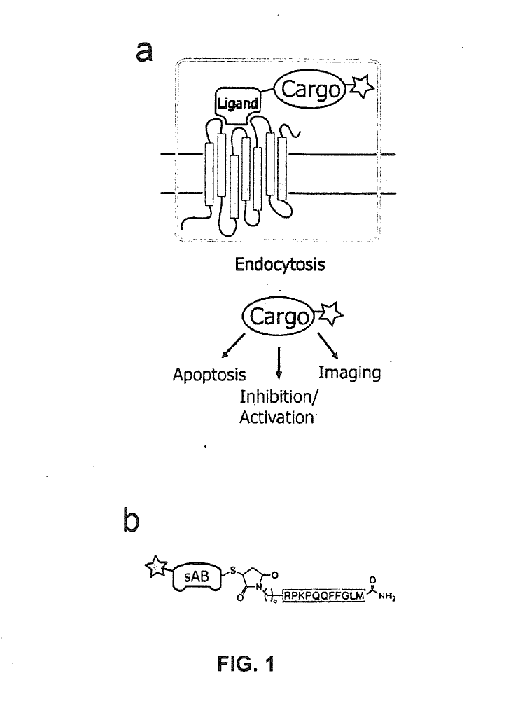

Cellular ligand-induced receptor internalization pathways are utilized in the invention to provide methods and compositions capable of efficient transmembrane delivery of a diverse set of agents including polynucleotides and polypeptides. Attachment of the agent to a ligand of a tachykinin receptor, e.g., neurokinin 1 (NK-1), allows delivery of the agent by receptor-mediated delivery (RMD) to the cytoplasm, where the agent can perform a desired function without compromising the integrity of the membrane (FIG. 1a). Additionally, by utilizing receptors that are up-regulated during disease states, the invention provides a powerful drug delivery tool that specifically targets diseased cells without affecting the surrounding normal tissue.

The invention provides both compositions and methods useful in delivering an agent to a cell expressing a tachykinin receptor, including an NK-1, NK-2 or NK-3 receptor by the ligand. As will be appreciated, the invention is useful in imaging intracellular proteins, targeting intracellular structures, and delivering moieties such as polypeptides, polynucleotides and small molecules or chemicals to cells for diagnostic, therapeutic and research purposes. Accordingly, delivery of agents in vivo, ex vivo and in vitro is contemplated.

In some embodiments, the methods of the invention utilize SP, an 11-amino acid peptide that is rapidly internalized upon binding to the NK-1 receptor, to deliver an agent into a cell. A composition including SP or a variant thereof that can be linked to an agent is also provided. The SP used in the composition is engineered as described herein so that the SP can be easily conjugated to virtually any protein with a surface cysteine or a reactive amine group for delivery to the cytoplasm. In particular embodiments, the invention provides a method for the delivery of a biologically active agent. For example, a set of synthetic antibody fragments (sABs) engineered by phage display to target various forms of actin was used in the examples to test the ability of the conjugates to deliver agents to an intracellular target. By conjugation to the synthetic SP composition, the actin-binding sABs were rapidly internalized by HeLa cells expressing the NK-1 receptor. The internalized conformationally specific sABs retained their in vitro properties within the cytoplasm, where they localized to and affected the polymerization of various forms of actin.

As used herein, “SP” refers to any member of a family of ligands that includes neurotransmitter peptides such as substance P (SP), neurokinin (NKA), neurokinin B (NKB) and hemokinin-1 (HK-1). (NM Page, J. Peptides 26: 1356-68 (2005), incorporated herein by reference in its entirety.) The cognate receptors for these ligands are a family of G-protein coupled receptors, namely NK-1, NK-2 and NK-3. The relative affinity of the tachykinin ligands for each of these receptors varies, however, binding occurs in each instance to some degree. By definition, the tachykinins are short peptides which share a conserved hydrophobic C-terminal region, FXGLM-NH₂ (SEQ ID NO: 7), where X is always a hydrophobic residue that is either an aromatic or a beta-branched aliphatic. Thus, although the invention is described with respect to a particular embodiment, i.e., SP/NK-1 receptor-mediated delivery, it is hypothesized that other combinations of the tachykinin ligands and receptors may be used in accordance with the invention.

Delivery to any cell expressing a tachykinin receptor is contemplated. In preferred embodiments, the cells express NK-1. Two NK-1 receptor isoforms have been reported in humans: a full length version and a truncated version that lacks 100 residues in its cytoplasmic tail. NK-1 receptors are natively expressed on nervous tissue (both central and peripheral) as well as non-neuronal cells of the immune, inflammatory and vascular endothelial systems. Thus, delivery of agents to any of these tissues is within the scope of the invention. Tachykinin receptors have been identified in a number of cells and tissues, including the peripheral and central nervous system, and inflammatory cells such as macrophages, lymphocytes, neutrophils, dendritic cells and mast cells. In addition, NK-1 receptors have been reported to be overexpressed in glioblastomas, astrocytomas, adenocarcinomas and breast, gastric, laryngeal and pancreatic cancers (Schultz et al., J. Histochemistry & Cytochemistry 54 (9): 1015-1020 (2006), incorporated herein in its entirety). The invention is therefore particularly suitable for specific delivery of an agent to neoplastic tissues expressing overexpressing NK-1 receptors. Moreover, cells that do not natively express tachykinin receptors may be engineered to express tachykinin receptors using techniques known in the art, thereby providing a means by which agents can be delivered to the engineered cells in accordance with the invention. For example, a polynucleotide encoding a NK-1 receptor operably connected to a promoter functional in the engineered cell may be introduced into the cell. Those of skill in the art are familiar with a variety of techniques for introducing polynucleotides into cells, including transfection and transduction.

The NK-1 receptor is highly overexpressed in a number of aggressive tumors, particularly in astrocytomas and glioblastomas, where the level of expression is correlated with the degree of malignancy. In the context, conjugates internalized by U87 glioblastoma cells, but not by normal human astrocytes (NHA) are preferred. As an illustration, in the Examples, a synthetic antibody or “sAB” that disrupts the structure of actin filaments was shown to decrease the viability of glioblastoma cells upon conjugate-mediated internalization. These data suggest that the delivery of sABs may have applications as a research tool and also may mediate the delivery of highly specific therapeutic affinity reagents that specifically target intracellular components within tumor cells.

Agents which can be delivered to cells in accordance with the invention include any chemical or biological entity that may be internalized, e.g., via endocytosis, into a cell via a tachykinin receptor when coupled to a targeting vehicle including SP. Suitable examples include, but are not limited to polynucleotides such as coding sequences and RNAi inducing agents (e.g., short interfering RNA (siRNA), short hairpin RNA (shRNA) and antagomirs), polypeptides such as antibodies or fragments thereof (e.g., fragment antigen binding domains (or “Fabs”) and sAB), normal cellular proteins such as myosin or mutant proteins (e.g., recombinant proteins which act as activators or inhibitors (e.g., dominant negative inhibitors), inside the cell), chemicals including cytotoxic moieties such as toxins, imaging moieties such as radioisotopes and fluorescent tags, and nanoparticles. Agents to be delivered are conjugated or coupled to SP to form a conjugate. Methods of coupling SP to agents are described below. Delivery of the agent to a cell expressing the NK-1 receptor includes contacting the cell with the conjugate. The cell may be contacted with the conjugate in vivo, in vitro or ex vivo.

In some embodiments, a polynucleotide comprising a sequence encoding a polypeptide operably linked to a promoter may be delivered to cells according to the methods of the invention. The polynucleotides may be provided as linear double-stranded DNA, or may be associated with vectors, e.g., plasmid vectors. In the Examples, a polynucleotide encoding GFP was conjugated to SP and 24 hours later GFP was detected by fluorescence microscopy.

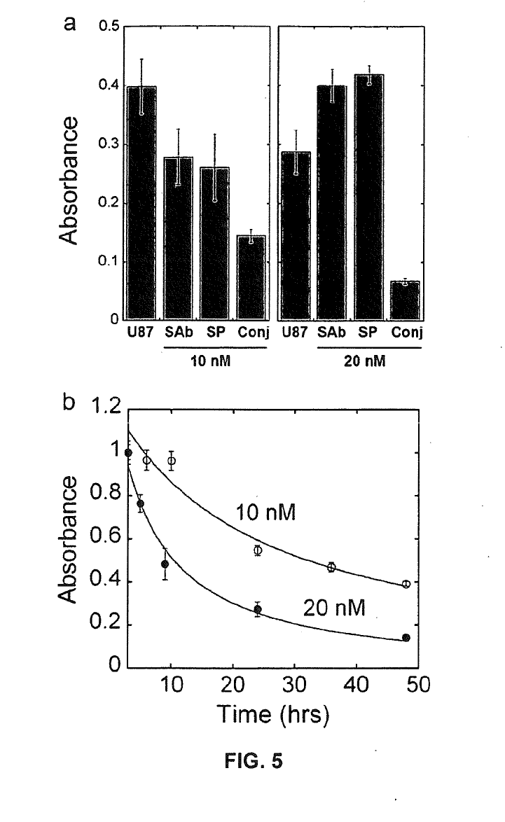

In an additional embodiment, a cytotoxic moiety may be delivered. A “cytotoxic moiety,” as used herein, refers to any agent which induces apoptosis, anoikis, autophagy, or ecrosis in cells to which the cytotoxic moiety is delivered. Cytotoxic moieties may, for example, interfere with cell division, disrupt normal functionality of microtubules, inhibit utilization of a metabolite, substitute nucleotide analogs into cellular DNA, or inhibit enzymes which are necessary for DNA replication. For example, a sAB which binds to actin and ultimately inhibits cell division may be suitably delivered as a cytotoxic agent. It has been found that sAB27 induces a twist in filaments which results in bundling actin filaments. In the Examples, sAB27 was conjugated to SP and delivered to U87 glioblastoma cells. As shown in FIG. 5, 48 hours after delivery the proliferation of cells treated with sAB27 was significantly decreased as compared to controls treated with SP or sAB27 alone.

In certain embodiments, imaging agents are delivered to a cell comprising an NK-1 receptor. As used herein, an “imaging agent” is any entity which enhances visualization or detection of the cell to which it is delivered. Imaging agents can include chemical entities that comprise an unstable isotope, i.e., a radionuclide or a fluorescent moiety, such as Cy-5. Suitable radionuclides include both alpha- and beta-emitters. In some embodiments, the targeting vehicle is labeled. In other embodiments, suitable radioactive moieties include labeled polynucleotides and polypeptides which can be coupled to the targeting vehicle. Radionuclides emitting low-energy electrons (e.g., those that emit photons with energies as low as 20 keV) are particularly suitable for some embodiments of the invention because they can irradiate the cell to which they are delivered without irradiating surrounding cells or tissues. Non-limiting examples of radionuclides that are can be delivered to cells in accordance with the invention include ¹³⁷Cs, ¹⁰³Pd, ¹¹¹In, ¹²⁵I, ²¹¹At, ²¹²Bi and ²¹³Bi, among others known in the art. In the Examples, sAB4, was labeled with Cy-5 to track internalization of the agent.

Further imaging agents suitable for delivery to a cell in accordance with several embodiments of the invention include paramagnetic species for use in MR1 imaging, echogenic entities for use in ultrasound imaging, fluorescent entities for use in fluorescence imaging (including quantum dots), and light-active entities for use in optical imaging. Specific non-limiting examples of fluorophores that may be delivered to a cell include, e.g., Cy-3, fluorescein and rhodamine. A suitable species for MRI imaging is a gadolinium complex of diethylenetriamine pentacetic acid (DTPA). For positron emission tomography (PET), ¹⁵F or ¹¹C may be delivered.

In another embodiment, the agent to be delivered to the cell expressing the NK-1 receptor is an antibody or fragment thereof such as a Fab fragment or a sAB. The antibody or fragment thereof may suitably recognize a particular intracellular protein, protein isoform, or protein configuration. Suitable Fab fragments include, but are not limited to, synthetic antibodies sAB10, sAB19 or sAB27. Antibodies or fragments thereof may inhibit or activate an intracellular function or protein. For example, sAB27 inhibits actin polymerization.

The above-described agents are suitably coupled to a targeting vehicle to form a conjugate for delivery of the agent to a cell comprising an NK-1 receptor as a conjugate. A “targeting vehicle” as used herein, refers to an entity that binds a tachykinin receptor and induces receptor-mediated endocytosis of the agent, regardless of whether downstream biological effects of tachykinin receptor binding are observed. As will be appreciated, no particular level of binding specificity is required, and acceptable levels of specificity will depend on the application. The targeting vehicle may be modified to include at least one reactive group, including, but not limited to, cysteine and malcimidc. Selection of a suitable targeting vehicle will depend on, e.g., the agent to be delivered, the coupling strategy to be used, and the degree of receptor activation desired, if any.

Conjugates include a targeting vehicle and an agent and have the following structure: P-M-C-A. P is a peptide ligand of a tachykinin receptor or a variant thereof, such as Substance P. P includes the seven amino acid NK-1 receptor binding region of SEQ ID NO: 1. In one embodiment, P includes the variant SP of SEQ ID NO: 2. In an alternative embodiment, P includes the K3C amino acid substitution shown in SEQ ID NO: 3. In this embodiment, the agent may be directly linked to P via a disulfide bond to a cysteine in a polypeptide agent or a sulfhydryl group in a chemical agent. P may also include any of SEQ ID NO: 4-6. M is maleimide or other reactive group attached to P. Suitably M is attached to or near the N-terminus of P. C is an optional crosslinker to attach M to A. Suitable crosslinkers are available to those skilled in the art. A is an agent attached to M either directly or via a crosslinker, C. Suitably, A is attached to maleimide via a thioester bond with a cysteine residue or other sulfhydryl group in/on the agent.

Suitable means of coupling an agent to a targeting vehicle include those, as described in, e.g., Bioconjugate Techniques, Greg T. Hermanson (1996) Academic Press, San Diego, incorporated herein by reference. Disulfide coupling using cysteine residues outside the binding domain of the SP to the NK-1 receptor is one particularly preferred coupling strategy. In particular embodiments, one or more cysteine residues or a malcimidc may be incorporated in the targeting vehicle to facilitate coupling to the agent to be delivered. Additionally, one or more cysteine residues may be introduced in the agent to facilitate coupling to the targeting vehicle. In some embodiments, the coupling strategy is chosen to facilitate release of the agent from the targeting vehicle upon uptake by the cell, however, in other embodiments, this is not necessary to achieve the desired effect.

In one formulation, SPDP (N-succinimidyl 3-(2-pyridyldithio)propionate), commercially available, hetero-bifunctional cross-linking reagent, is used to couple a nucleic acid agent, e.g., DNA or siRNA, to a targeting vehicle. As is appreciated by those skilled in the art, one end of SPDP contains an activated N-hydroxysuccinimide (NHS) ester, which reacts with alkyl amine groups to form an amide linkage. The other end of SPDP contains a 2-pyridyldithiol group, which reacts with sulfhydryl groups (—SH) to form a disulfide linkage. The nucleic acid agent is suitably prepared to provide a hexylamine moiety at one of its termini. Conjugation to the targeting agent occurs in two stages. In the first stage, the hexylamine group reacts with the NHS moiety of SPDP to form a nucleic acid-SPDP conjugate. In the second stage, the 2-pyridyldithiol group of this conjugate reacts with a cysteine residue in the targeting vehicle via thiol disulfide exchange chemistry, generating the targeting vehicle-nucleic acid conjugate.

In a second strategy, a different hetero-bifunctional cross-linking reagent, SMCC (succinimidyl 4-(N-maleimidomethyl)cyclohexane-1-carboxylate), is used. Like SPDP, one end of SMCC contains an activated NHS ester, but the other end contains a maleimide group, which reacts with thiols. The strategy for coupling this reagent parallels the SPDP-based strategy described above. In stage 1, the NHS ester eacts with the hexylamine-containing nucleic acid, and in stage two, the resulting nucleic acid-SMCC conjugate reacts with a cysteine residue in the targeting vehicle, such as in the SP of SEQ ID NO: 3. In the SPDP linked conjugate, the targeting vehicle is attached to the nucleic acid via a disulfide bond, which may be reduced in the intracellular environment. In the SMCC linked conjugate, the targeting vehicle is attached to the nucleic acid via a thioether linkage, which is inert under physiological conditions.

In addition to the disulfide exchange chemistry and maleimide strategies described above, there are numerous other chemistries that are suitable for coupling agents to targeting vehicles, including, but not limited to: haloacetyl and alkyl halide derivatives, aziridines, acryloyl derivatives and arylating agents.

In the methods of the invention, delivery of an agent to a cell expressing a tachykinin receptor is affected by contacting the cell with the conjugate described above. It is presently believed that the P portion of the conjugate binds to the tachykinin receptor on the cell and mediates internalization of the conjugate by receptor-mediated delivery. In some embodiments, the agent is then transported out of the endosome to an intracellular, extra-endosomal location. The agent may remain in the cytoplasm, or may be transported to the nucleus or a sub-cellular compartment in the cell. In certain embodiments, the agent is then capable of interacting with the appropriate target within the cell. Suitable targets within the cell include, but are not limited to, cellular proteins, cellular organelles, mRNA, micro RNA, DNA and intracellular pathogens. The agent may be capable of inhibiting the target, activating the target or labeling the target without affecting its normal function within the cell. In some embodiments, the effect of the agent on the target causes the cell to stop proliferating and induces cell death.

Also provided are methods of arresting cell growth or inducing cell death of a cancer cell expressing a tachykinin receptor. The methods include contacting the cancer cell with a conjugate capable of mediating intracellular delivery of an agent, such as the conjugates described herein. The agent is capable of arresting or attenuating the growth of the cell or inducing cell death through any mechanism after agent internalization. The cancer cell may be contacted with the conjugate in vitro, in vivo or ex vivo. These methods may be useful in treating cancer by directly targeting cancer cells overexpressing tachykinin receptors for delivery of agents capable of decreasing or arresting cell growth or inducing cell death.

### EXAMPLES

### Example 1

### Materials and Methods

Peptide synthesis of SP variants: All protected amino acids and resin for peptide synthesis were purchased from Peptides International, solvents from Fisher Scientific, salts and buffers from Sigma Aldrich. Substance P-maleimide synthesis was carried out manually by t-Boc methods using p-Methyl-Benzhydrylamine resin to produce a C-terminal amide after HF cleavage. 6-maleimidohexanoic acid (Sigma) was used to introduce an N-terminal maleimide with a 6-carbon linker. Additionally, a variant of SP was synthesized with a cysteine mutation at position 3 for attachment of thiol-reactive fluorophores and was used as positive control for NK-1 receptor-mediated internalization. The peptides were cleaved from the resin using HF and extracted in 50% acetonitrile, 0.1% trifluoroacetic acid aqueous solution for lyophilization. The purity of the crude peptides was determined using an analytical C-18 reversed-phase column on a Shimatzu 10A-vp and their masses were confirmed by MALDI MS.

Mutagenesis: Alanine 121 in heavy chain was mutated to cysteine of actin sAB constructs was introduced by Kunkel mutagenesis. The new construct was transformed into 55344 bacteria expression strain (provided by Genentech). Actin-binding sABs were engineered by phage display and purified. Each sAB contained a C-terminal 6-his tag on the light chain, and a cysteine mutation at position Ala121 of the heavy chain, and sequences were confirmed by DNA sequencing. The purified sABs were dialyzed into 50 mM Sodium Borate buffer pH 7.4 for fluorescent conjugation. NHS-ester fluorophores (Cy3 or Cy5; GE Healthcare, Alexa-488 or Alexa-647; Invitrogen) were added to each sAB in a 5-fold excess from a 10 mM stock solution in DMSO. Reactions were carried out at room temperature for 1 hr, followed by gel filtration using a PD-10 column (GE Healthcare) equilibrated with 20 mM MOPS, 100 mM NaCl pH 6.9 to remove excess fluorophore. The fluorescently-labeled sABs were reacted with a 10-fold excess of SP-maleimide targeting vehicle from a 5 mM stock in DMSO. The reaction was carried out overnight at 4° C. in 20 mM MOPS, 100 mM NaCl pH 6.9, followed by gel filtration using a PD-10 column to remove excess targeting vehicle.

Conjugation of SP to full-length antibodies: Polyclonal anti-actin antibodies were purchased from Cell Signaling Technology. Antibodies were fluorescently labeled and crosslinked simultaneously by addition of 5-fold molar excess of NHS-ester Alexa-555 and SPDP (Thermo) in 50 mM Sodium Borate pH 7.4 for 1 hr at room temperature, followed by PD-10 column gel filtration. The modified antibodies were incubated with a 10-fold excess of targeting vehicle (SP-maleimide) overnight at 4° C. in 20 mM MOPS, 100 mM NaCl, 1 mM TCEP, pH 6.9, followed by gel filtration using a PD-10 column.

Cell lines: U87 human glioblastoma-astrocytoma epithelial-like cells (provided by Prof. Maciej S. Lesniak, University of Chicago Medical Center) were cultured in MEM with penicillin/streptomycin and 10% FBS. Normal human astrocytes (NHA) were purchased from Lonza and cultured in astrocyte growth media (Lonza) supplemented with rhEGF; Insulin, Ascorbic Acid, GA-1000 L-Glutamine and 5% FBS. HeLa cells (provided by Dr. Ronald Rock, University of Chicago) were grown in DMEM penicillinistreptomycin and with 10% FBS.

Western blot: NHA and U87 cells were lysed in RIPA buffer (50 mM Tris-HCl, pH 7.4, 150 mM NaCl, 1 mM PMSF, 1 mM EDTA, 1% Triton X-100, 1% Sodium deoxycholate, 0.1% SDS, 5 μg/μL proteinase inhibitors). Total protein concentrations of the lysates were determined. For each sample, 20 μg protein was loaded on 4-20% Tris-HCl gradient gel for separation by SDS-PAGE electrophoresis. The proteins were transferred to Hybond-C Extra nitrocellulose membrane at 90 V for 90 minutes. The membrane was blocked for 1 h in 5% milk in TBS-Tween and incubated overnight at 4° C. with rabbit anti-NK-1R antibody (1:2000), then incubated with HRP-conjugated anti-rabbit antibody (1:2000) for 1 hour at room temperature. ECL Western Blotting Detection Reagent (Amersham) was used for chemifluorescence signal detection.

NK-1 receptor transfection: A plasmid coding for the human NK-1 receptor in the mammalian expression vector pcDNA3(+) (provided by Dr. Robert Lefkowitz, Duke University) was transfected into HeLa cells using Lipofectamine 2000 according to manufacturer protocol, with 1 ng of the plasmid for each well containing 10⁴ HeLa cells seeded on an 18 mm cover-slip in a 12-well plate.

Confocal Microscopy: For each sample, 10⁴ U87 cells were seeded on an 18 mm cover-slip in MEM with 10% FBS overnight in a 12-well plate, then starved in MEM with 0.1% BSA for 3 hrs. The reagent (SP-sAB conjugate, SP-Fluorophore or sAB only) was filter-sterilized, diluted to the desired final concentration in MEM 0.1% BSA and added directly to the live cells in each well for 2 hrs. Untreated cells served as a negative control. Following incubation with the reagent, the cells were washed with PBS and fixed in 4% p-formaldehyde. The cells were permeablized in TBS-Tween and blocked with 10% Normal Goat Serum for 1 hr. Actin filaments were stained with FITC-phalloidin (1:250 dilution), or TRITC-phalloidin (1:3000 dilution) for 1 hr at room temperature. Nuclei were stained with DAPI (10 μg/mL) or Hoechst reagent (Invitrogen, 1:2000) for 5 minutes at room temperature. Samples were mounted using ProLong Gold reagent (Invitrogen). Confocal microscopy images were collected using a Leica SP2 with Alexa 488, Alexa 555 or Cy3 and Alexa 574 or Cy5 excited with the 488 nm laser, the 543 nm laser and the 633 laser, respectively. Multicolored images were overlaid using the imageJ software.

MTS proliferation assay: U87 cells were seeded in 96-well plates at density 5000 cells/well in 0.1 mL of growth media. Cells were attached overnight and starved for 3 hours. Cells were stimulated with 20 nM sAB27-SP conjugate for 3, 5, 9, 24 and 48 hours, and SP or sAB27 alone for 48 hours in starving media. 20 μL of the MTS-based CellTiter 96 Aqueous One Solution reagent was added to each well. The plate was incubated in a humidified, 5% CO₂ atmosphere until color was developed and the absorbance was recorded at 490 nm.

Internalization quantification: U87 cells were seeded in a 96-well black bottom plate at a density of 5000 cells/well in 0.1 mL growth media, and then starved in starving media for 3 hours. Cells were stimulated with 20 nM, 50 nM, 100 nM, 200 nM and 500 nM of Cy5-sAB-27-SP conjugate for 24 hours in starving media. The same concentrations were added to empty wells to account for non-specific binding of the conjugate. After 24 hours, the empty wells and cells were extensively washed with PBS in order to remove non-internalized conjugate. 0.1 mL of PBS was added to each well and the remaining fluorescence was measured in triplicates using the following parameters: excitation: 640 nm, emission wavelength 670-680 nm, excitation bandwidth: 8 nm, emission bandwidth: 15 nm. A standard curve was used to calculate the concentration of internalized conjugate based on fluorescence measurements.

### Example 2

### Engineering a Delivery Vehicle Based on SP

An important feature of SP and variants thereof is that the seven amino acids at the C-terminus are sufficient for binding to the tachykinin receptor and subsequent internalization. This allows the incorporation of modifications for agent attachment at the N-terminus without loss of function. We synthesized a variant of SP that contains a C-terminal amide, in addition to an N-terminal thiol-reactive maleimide with a 6-carbon linker (FIG. 1b) using t-Boc chemistry. The maleimide moiety reacts with cysteine residues under physiologic conditions to form a stable thioether bond.

### Example 3

### Construction of sAB Conjugates

To demonstrate the ability of engineered SP to delivery protein agents, we isolated three sABs (sAB-4, sAB-17 and sAB-27) from a restricted amino acid phage display library that had been engineered to bind to the pointed end of actin. Characterization of the actin-binding properties of the sABs indicated that each exhibits a unique binding mode to the actin filaments. sAB-4 causes rapid depolymerization of actin filaments, sAB-17 is a side-binder that has no apparent effect on the actin filament structure, and sAB-27 affects the overall structure of actin by inducing a twist in the filament. Prior to conjugation of the sABs to SP, each sAB was fluorescently labeled via surface lysines using an amine reactive fluorophore to facilitate visualization by confocal microscopy. As a consequence of the restricted amino acid library used for the sAB selection process, lysine residues are rarely incorporated in the antigen binding site. We found that targeting surface lysines for fluorescence labeling has little to no effect on the actin-binding properties of the sABs. A cysteine mutation was introduced at position 121 of the heavy chain of each sAB for conjugation to the N-terminal maleimide of our SP variant (FIG. 1b). Position 121 was chosen because it is solvent accessible and lies far from the antigen binding site.

### Example 4

### NK1 Receptor-Mediated Delivery of sAB Conjugates

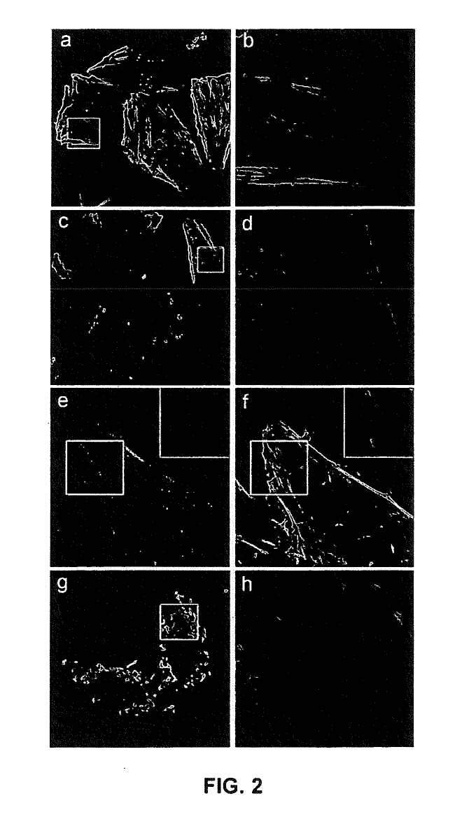

The ability to specifically deliver functional biomolecular agents to living cells can be a powerful research tool for studying molecular processes. Introduction of the NK-1 receptor is sufficient for the uptake of fluorescently-labeled SP. We sought to demonstrate that attachment of sABs to SP does not impede uptake by the receptor, and further, that the attached sABs retain their activity within the cells after internalization. HeLa cells transfected with a plasmid coding for the NK-1 receptor were incubated for 2 hours with 20 nM fluorescently-labeled SP. The cells were then stained with phalloidin for visualization of actin structure. Confocal microscopy imaging of the cell shows rapid internalization of SP by cells transfected with the receptor. Each of the three SP-conjugated sABs were also internalized by the transfected cells in as little as 2 hrs, demonstrating the ability of the engineered SP variant to serve as a delivery vehicle for protein agents (FIG. 2).

While the internalized SP shows no specific localization with actin and no apparent effect on the structure of the actin filaments (FIGS. 2a, b), treatment of transfected HeLa cells with 20 nM of each of three SP-sAB conjugates shows localization or reorganization of actin structure. Cells treated with 20 nM of the sAB-4-SP conjugate display the presence of short actin filaments in many cases capped by the sAB (FIGS. 2c, d), suggesting that this sAB induces severing or depolymerization of the filaments in the cytoplasm as observed by in vitro characterization. The cells also exhibit poor staining with phalloidin, which does not bind effectively to short actin filaments. In contrast, cells treated with 20 nM of the sAB-17 conjugate show co-localization of the sAB with actin filaments without an apparent change in actin structure or cell shape (FIGS. 2e, f). This sAB was determined to be a “side-binder” by in vitro characterization, and consequently, the binding pattern resembles that of a phalloidin stain. Finally, internalization of sAB-27 induces a conformational change to the actin filaments and possibly stimulates bundling that drastically changes the overall shape of the cells (FIGS. 2g, h). Similar results are obtained at very low concentrations (2 nM) of sAB-27 suggesting a long-range effect of the sAB-actin interaction.

### Example 5

### Specific Delivery of sABs to Tumor Cells

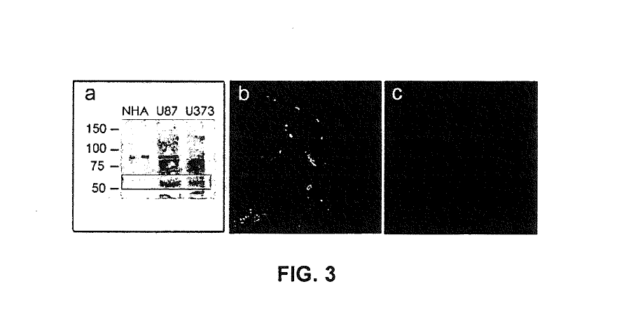

The NK-1 receptor is highly overexpressed in many tumor cell lines and primary cancers, including breast carcinomas, adenocarcinomas of the colon, astrocytomas and glioblastomas suggesting therapeutic potential for the compositions and methods of the invention. Further, the expression level of the NK-1 receptor is correlated with the degree of malignancy of brain tumors. We compared the human glioblastoma cell-lines U87 and U373 with normal human astrocytes (NHA) for expression of the receptor in an effort to expand the utility of SP-mediated delivery as a specific tool for targeting tumor tissue. Western blot analysis of the NK-1 receptor expression confirms the presence of a significant level of the receptor in both U87 and U373 cells, whereas the receptor expression level is undetectable in NHA (FIG. 3a). In addition, U87 glioma cells and NHA were incubated with 200 nM Alexa555-labeled SP for 2 hr, followed by DAPI staining. Confocal microscopy imaging shows that U87 cells readily internalize the fluorescent SP conjugate (FIG. 3b), where internalization is undetectable in normal astrocytes (FIG. 3c).

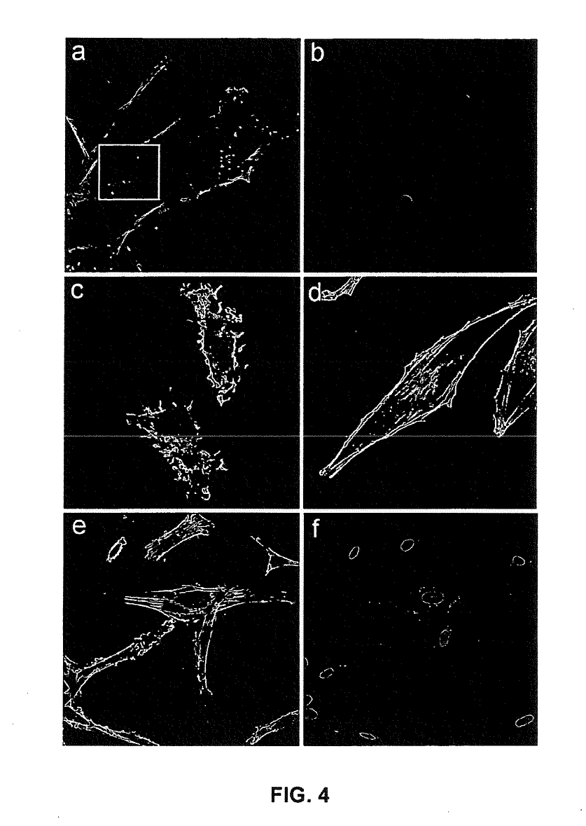

We investigated the ability of SP to deliver functional proteins to tumor cells. Live U87 glioblastoma cells were treated for 2 hrs with 20 nM of the sAB4- or sAB27-SP conjugates, and phalloidin staining was used to determine the localization of the internalized sABs by confocal microscopy. The results indicate that U87 cells treated with the SP-sAB conjugates retain their actin-remodeling function. As observed in HeLa cells transfected with the NK-1 receptor, sAB-4 localizes with short actin filaments (FIGS. 4a, b), and sAB-27 disrupts both the structure of the actin filaments and the overall shape of the cells (FIG. 4c) compared to untreated U87 cells (FIG. 4d). Similar results were obtained when a full length actin antibody was conjugated to SP and incubated with U87 cells as shown in FIG. 4e-f.

### Example 6

### The Effect of the sAB-27 on Cell Viability

Due to the actin-bundling activity of sAB-27, its effect on the viability of U87 glioblastoma cells following SP-mediated delivery was investigated. Live U87 cells were treated with either 10 nM or 20 nM of the SP-sAB-27 conjugate and a cell viability assay was carried out at different time points up to 48 hrs after treatment. The viability of cells incubated with the conjugates was compared with that of cells treated with SP alone or sAB-27 alone, as well as untreated cells. Treatment with SP or sAB-27 alone has no effect on viability; however, incubation of U87 cells with the SP-sAB-27 conjugate results in a concentration-dependent decrease in cell viability after 48 hrs (FIG. 5a). A time-course of the viability of U87 cells treated with the SP-sAB-27 conjugate was carried out, demonstrating the ability of this conjugate to decrease the viability of the glioblastoma cells within 24 hrs following treatment (FIG. 5b).

### Example 7

### Quantitative Analysis of SP-Mediated Internalization of sAB-27

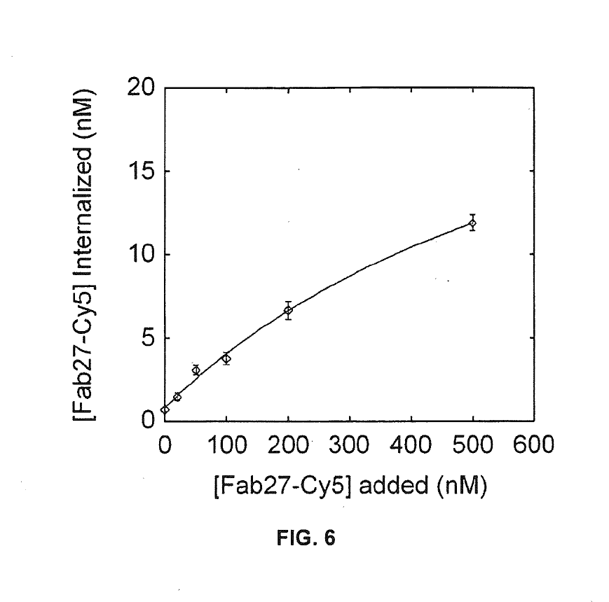

To determine the efficiency of SP-mediated delivery of proteins, U87 cells were incubated with increasing concentrations of a cy5-labeled sAB-27-SP conjugate for 24 hrs in a 96-well plate. The cells were washed extensively with PBS and residual fluorescence was determined using a microplate reader. The fluorescence values obtained, reflecting the amount of internalized conjugate, were compared to the input concentrations (FIG. 6). The results show a correlation between the amount of conjugate added and the concentration of the internalized conjugate that fits a hyperbolic response. The fit suggests an upper limit to the ability of U87 cells to internalize the SP-conjugates with saturation at high concentrations (>1 μM) of added conjugate. This apparent limit may be due to down-regulation of the NK-1 receptor as a result of stimulation by SP. Additionally, it has been suggested that concentration of cell-surface NK-1 receptor can vary between malignant cell lines and can be influenced by extracellular factors secreted by the immune system. We also observed that the ability of cells to internalize SP can diminish at high passage numbers, presumably due to the absence of immune factors from artificial culture conditions.

### Example 8

### Delivery of a Polynucleotide Encoding a Fluorescent Protein

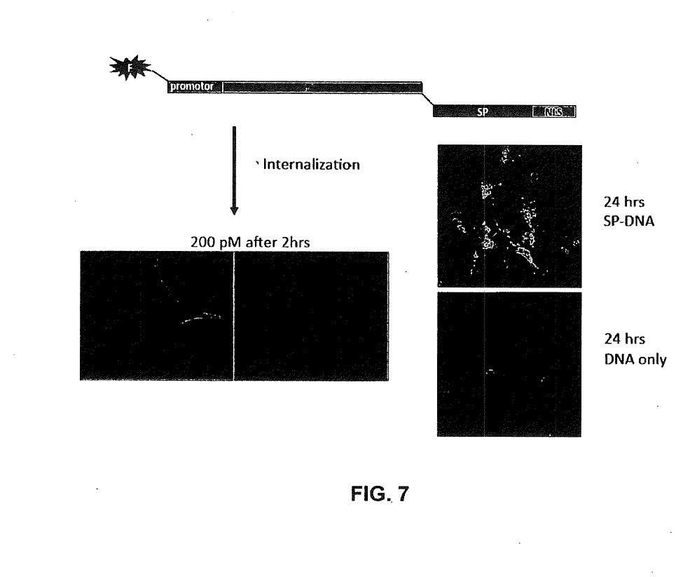

To determine if other types of agents could also be delivered to cells expressing NK-1 receptors in accordance with the invention, HeLa cells transfected with the NK-1 receptor as described above, were incubated with an ST-polynucleotide conjugate. The polynucleotide encoded GFP operably linked to a promoter functional in the cells. The polynucleotide was also fluorescently tagged with Alexa-647 such that delivery of the polynucleotide into the cell could be monitored as well as expression of GFP. As shown in FIG. 7, in cells incubated with the 200 pM SP-polynucleotide conjugate, the polynucleotide was internalized after 2 hours and at least a portion co-localized with the nucleus of the cell (red). Confocal microscopy of cells incubated with the 200 μM SP-polynucleotide conjugate or the polynucleotide alone show expression of GFP after 24 hours only in cells incubated with the SP-polynucleotide conjugate (FIG. 7). These results demonstrate that polynucleotide as well as polypeptide agents may be delivered via this method and that polynucleotides can be further targeted to the nucleus.

### Example 9

### Delivery of Myosin

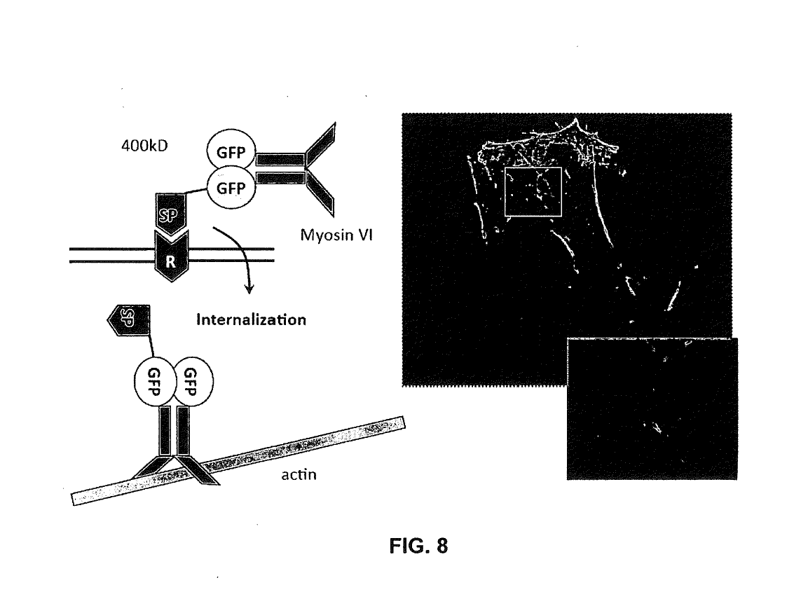

To determine whether large agents such as full-length proteins could be delivered by way of the invention, a conjugate in which SP was conjugated to a GFP-myosin VI chimera was developed. The resulting conjugate is 400 kDa. U87 cells were incubated with the conjugate for 2 hours and internalization was visualized using confocal microscopy. As shown in FIG. 8, the conjugate was internalized by the cells. Indeed, the myosin (red) was functional and could be visualized walking on actin filaments within the cells. Thus the methods are useful for delivering even very large agents to cells expressing tachykinin receptors such as the NK-1 receptor.

It is specifically contemplated that any embodiment of any method or composition of the invention may be used with any other method or composition of the invention.

As used in this specification and the appended claims, the singular forms “a,” “an,” and “the” include plural referents unless the content clearly dictates otherwise. Thus, for example, reference to a composition containing “a conjugate” includes a mixture of two or more conjugates. It should also be noted that the term “or” is generally employed in its sense including “and/or” unless the content clearly dictates otherwise.

It also is specifically understood that any numerical value recited herein includes all values from the lower value to the upper value, i.e., all possible combinations of numerical values between the lowest value and the highest value enumerated are to be considered to be expressly stated in this application. For example, if a range is stated as 1% to 50%, it is intended that values such as 2% to 40%, 10% to 30%, or 1% to 3%, etc., are expressly enumerated in this specification.

1. A conjugate having the structure:
P-M-C-Ab,
wherein P is a tachykinin receptor ligand or a variant thereof; M is a reactive moiety attached to or near the N-terminus of P; C is an optional crosslinker; and Ab is an antibody or fragment thereof specific for an intracellular target and is attached to M or C, if present.

2. The conjugate of claim 2, wherein the intracellular target is an extra-endosomal target.

3. A conjugate having the structure:
P-M-C-A,
wherein P is a tachykinin receptor ligand or a variant thereof; M is a maleimide moiety attached to or near the N-terminus of P; C is an optional crosslinker; and A is an agent attached to M or C, if present.

4. The conjugate of claim 1, wherein P is an NK-1 receptor ligand.

5. The conjugate of claim 1, wherein P comprises a ligand selected from the group consisting of SEQ ID NO: 1, SEQ ID NO: 2, SEQ ID NO: 3, SEQ ID NO: 4, SEQ ID NO: 5, and SEQ ID NO: 6.

6. The conjugate of claim 1, wherein M is maleimide.

7. The conjugate of claim 3, wherein A is a polynucleotide, a polypeptide, a chemical, or a nanoparticle.

8. The conjugate of claim 7, wherein the polynucleotide is an RNAi inducing agent, a coding sequence or a plasmid.

9. The conjugate of claim 7, wherein the polypeptide is selected from the group consisting of an antibody, a fragment of an antibody and a synthetic fragment of an antibody.

10. The conjugate of claim 7, wherein the polypeptide is a cytotoxic protein.

11. The conjugate of claim 7, wherein the chemical is selected from the group consisting of fluorescent tags, quantum dots, toxins, and imaging agents.

12. A method of delivering an agent to an intracellular, extra-endosomal location within a cell expressing a tachykinin receptor comprising contacting the cell with the conjugate of claim 1.

13. A method of delivering an agent to a cell in vitro, comprising:
(a) introducing a polynucleotide encoding an NK-1, NK-2 or NK-3 receptor into the cell, the polynucleotide being operably connected to a promoter functional in the cell; and
(b) contacting the cell with the conjugate of claim 1.

14. The method of claim 12, wherein the agent is delivered to the cytoplasm of the cell.

15. The method of claim 12, wherein the agent is delivered to the nucleus of the cell.

16. The method of claim 12, wherein the agent is delivered to a subcellular compartment of the cell.

17. The method of claim 12, wherein the agent is capable of inhibiting or enhancing the function of a target in the cell.

18. The method of claim 17, wherein the function of the target comprises attenuating cell proliferation or inducing cell death.

19. A method of delivering an antibody or a fragment thereof to a cell expressing a NK-1, NK-2 or NK-3 receptor comprising contacting the cell with the conjugate of claim 1, wherein the antibody or fragment thereof is capable of binding to an intracellular target in the cell.

20. The method of claim 19, wherein the target is extra-endosomal.

21. The method of claim 19, wherein the antibody or antibody fragment is a synthetic antibody fragment (sAB).

22. The method of claim 19, wherein the antibody or antibody fragment is capable of reducing, inhibiting or enhancing the function of the target.

23. The method of claim 22, wherein the function of the target comprises attenuating cell proliferation or inducing cell death.

24. A method of arresting cell growth or inducing death of a cancer cell comprising contacting the cancer cell with the conjugate of claim 1, wherein the agent is capable of attenuating cell growth or inducing death of the cancer cell.

25. The method of claim 24, wherein the cancer cell is selected from the group consisting of breast cancer, pancreatic cancer, gastric cancer, colon cancer, lung cancer, prostate cancer, ovarian cancer, laryngeal cancer, astrocytoma, and glioblastoma.

26. A method of delivering an agent to an intracellular, extra-endosomal location within a cell expressing a tachykinin receptor comprising contacting the cell with a conjugate having the formula:
P-M-C-A
wherein P is a tachykinin receptor ligand or a variant thereof; M is a reactive moiety attached to or near the N-terminus of P; C is an optional crosslinker; and A is an agent attached to M or C, if present.


SEQUENCE LISTING (7 sequences)

SEQ ID NO: 1 - protein, 7 aa, Homo sapiens
QQFFGLM

SEQ ID NO: 2 - protein, 11 aa, Homo sapiens
RPKPQQFFGLM

SEQ ID NO: 3 - protein, 11 aa, Homo sapiens
RPCPQQFFGLM

SEQ ID NO: 4 - protein, 7 aa, Homo sapiens
QQFFLPM

SEQ ID NO: 5 - protein, 10 aa, Homo sapiens
HKTDFFVGLM

SEQ ID NO: 6 - protein, 10 aa, Homo sapiens
DMHDFFVGLM

SEQ ID NO: 7 - protein, 5 aa, Homo sapiens
  misc_feature (2)..(2): Xaa can be any naturally occurring amino acid
FXGLM